(19) United States
(12) Patent Application Publication
(10) Pub. No.: US 2015/0284350 A1
(43) Pub. Date: Oct. 8, 2015

(54) VINYLIDENE DIMER DERIVATIVES

(71) Applicant: SHELL OIL COMPANY, Houston, TX (US)

(72) Inventors: Naga Roobini ARULESWARAN, Kajang Selangor DE (MY); Paul Theodore SHARKO, Houston, TX (US); James Grace CRUMP, Houston, TX (US)

(21) Appl. No.: 14/679,250

(22) Filed: Apr. 6, 2015

Related U.S. Application Data
(60) Provisional application No. 61/976,261, filed on Apr. 7, 2014.

(51) Int. Cl. C07D 307/20; C07D 301/12; C07C 303/02; C07C 303/20; C07C 45/58; C07C 41/03; C07C 213/04; C07C 2/86; C07C 29/00

(52) U.S. Cl. CPC C07D 307/20; C07C 2/865; C07D 301/12; C07C 303/02; C07C 29/00; C07C 45/58; C07C 41/03; C07C 213/04; C07C 303/20

(57) ABSTRACT

The present invention provides a process for producing vinylidene dimer derivatives, which process includes subjecting one or more vinylidene dimers to an epoxidation reaction to obtain a vinylidene-derived epoxide.

In another aspect the invention provides further process for producing vinylidene dimer derivatives.

### PRIORITY CLAIM

This non-provisional application claims the benefit of U.S. Provisional Application Ser. No. 61/976,261, filed Apr. 7, 2014.

### FIELD OF THE INVENTION

The invention relates to the derivatization of vinylidene dimers.

### BACKGROUND OF THE INVENTION

Alpha olefins or isomerized olefins have been used directly as precursors for chemicals suitable in applications such as consumer products like detergents, soaps, personal care products and pharmaceutical products, as well as industrial products such as surfactants, degreasers, industrial cleaners, agricultural adjuvants, textile processing chemicals, waxes, mining chemicals, oilfield chemicals, metal working fluids and additives for lubricating oils and greases. Examples of drilling fluids are described in U.S. Pat. No. 7,081,437 which describes a drilling fluid comprising a mixture of three internal olefin fractions.

It would be advantageous to provide an alternative to alpha olefins or isomerized olefins that have improved physical properties in particular with respect to pour point and viscosity.

### SUMMARY OF THE INVENTION

The invention provides vinylidene dimer alternatives to alpha olefins or isomerized olefins and derivatives of such vinylidene dimers.

Accordingly, the invention provides a process for producing vinylidene dimer derivatives, which process includes subjecting one or more vinylidene dimers to an epoxidation reaction to obtain a vinylidene-derived epoxide.

In another aspect, the invention provides a process for producing vinylidene dimer derivatives, which process includes subjecting one or more vinylidene dimers to a sulfonation reaction to obtain a vinylidene-derived β,γ-unsaturated sulfonic acid.

In a further aspect, the invention provides a process for producing vinylidene dimer derivatives, which process includes subjecting one or more vinylidene dimers to an alkylation reaction with an aromatic compound to obtain a vinylidene-derived di-substituted aromatic compound.

In still another aspect, the invention provides a process for producing vinylidene dimer derivatives, which process includes subjecting one or more vinylidene dimers to a reaction with a maleic anhydride (MALA) to obtain a vinylidene-derived alkylsuccinic anhydride.

### DETAILED DESCRIPTION OF THE INVENTION

The present invention relates to the use of vinylidene, also referred to as vinylidene dimers, as an alternative to alpha olefins and isomerized olefins. Vinylidene may be prepared by dimerizing one or more even numbered alpha olefins.

Vinylidene dimers provide stereospecificity for functionalization. Dimerization of one or more even numbered alpha olefins to produce one or more vinylidenes can provide a useful building block in a variety of applications. These include consumer products such as detergents, soaps, personal care products, drug products, as well as industrial products such as surfactants, degreasers, industrial cleaners, agricultural adjuvants, textile processing chemicals, waxes, mining chemicals, oilfield chemicals, metal working fluids and additives for lubricating oils and greases. The dimers can serve as precursors for hydroformylation, esterification, ethoxylation, sulfonation, alkylation with aromatic rings, oligomerization, epoxidation and a host of several other reaction pathways.

In addition, the vinylidene dimers may have a low pour point and low kinematic viscosity compared to alpha olefins and isomerized olefins making them particularly suitably for application at low temperatures and/or requiring a low viscosity, for instance in applications like additives for lubricating oils and greases, metal working fluids and surfactants in a variety of applications such as industrial cleaners, textile processing chemicals and oilfield chemicals.

Furthermore the vinylidene dimers and derivatives thereof may also provide advantages with respect to handling and storage.

A process for preparing vinylidene dimers from even numbered alpha olefins has been disclosed in WO2012054369, incorporated herein by reference.

A process for preparing vinylidene dimers from even numbered alpha olefins may comprise dimerizing even numbered alpha olefins to form vinylidenes. These vinylidenes are typically of the general structure 2-alkyl-1-alkene. Even numbered alpha olefins are defined as any alpha olefin having an even number of carbon atoms. The even numbered alpha olefins may include any even numbered alpha olefin with from 4 to 16 carbon atoms. The even numbered alpha olefins preferably comprise even numbered alpha olefins with from 6 to 10 carbon atoms. More preferred even numbered alpha olefins have 6 or 8 carbon atoms.

The dimerization may be carried out with a single even numbered alpha olefin or a blend of even numbered alpha olefins. When a single even numbered alpha olefin is used, it is preferably a C6, C8, C10 or C12 alpha olefin. When a blend of even numbered alpha olefins is used, any combination of even numbered alpha olefins may be used.

Physical properties of the final product are typically impacted by the starting materials selected, so the use of some even numbered alpha olefins will result in more preferred final products. Some examples of possible blends of even numbered alpha olefins are C4 with C8; C4 with C10; C4 with C12; C4 with C14; C6 with C8; C6 with C10; C6 with C12; C6 with C14; C8 with C10; and C8 with C12. Further it is possible to envision a blend of more than two even numbered alpha olefins that could be used to produce suitable products.

The process will be described below in respect to using a single even numbered alpha olefin, C8, but this process applies equally to the other single even numbered alpha olefins and the blends of alpha olefins described above.

The process is to dimerize 1-octene to produce 2-hexyl-1-decene. The 2-hexyl-1-decene is a vinylidene olefin that may also be referred to as 7-methylene pentadecane. There are a number of processes for carrying out this dimerization; for example, the processes described in U.S. Pat. No. 4,658,078; U.S. Pat. No. 4,973,788; and U.S. Pat. No. 7,129,197, which are herein incorporated by reference. Dimerization using a metallocene catalyst results in a single vinylidene compound being formed. The product may be distilled, if desired, to remove unreacted monomer and any trimer or higher oligomers that may have formed or the product may be directly used in the next step.

The C16 and C20 vinylidene formed can be used as a base oil for a drilling fluid. Further, C16, C20, C22 and C24 vinylidenes can be blended with internal olefins, alpha olefins and/or paraffins for use in an oil based drilling fluid.

Suitable internal olefins include any internal olefin having a carbon number of from 10 to 18. For example, the internal olefins may be C15, C16, C17 or C18 internal olefins. These internal olefins may be used singly or in mixtures of more than one internal olefins. For example, the internal olefins blended with the vinylidene may comprise a mixture of C15, C16, C17 and C18 internal olefins.

Suitable alpha olefins include any alpha olefin having a carbon number of from 10 to 18. For example, the alpha olefins may be C16, C17, or C18 alpha olefins. These alpha olefins may be used singly or in mixtures of more than one alpha olefins. For example, the alpha olefins blended with the vinylidene may comprise a mixture of C16, C17 and C18 alpha olefins.

Suitable paraffins may be of mineral or synthetic origin. Synthetic paraffins may be formed by any process known to one of skill in the art, for example, a Fischer-Tropsch process. Suitable paraffins have a carbon number of from 10 to 24.

As can be seen in Table 1, the vinylidene products have improved pour point, flash point and viscosity properties compared to comparable alpha olefin and internal olefin products.

TABLE 1

| | | | C16 | C1618 |
|---|---|---|---|---|
| | C16 | C20 | alpha | internal |
| Property | Vinylidene | Vinylidene | olefin | olefin* |
| Appearance | Clear | Clear | Clear | Clear |
| | liquid | liquid | liquid | liquid |
| Pour point, ° C. | −42 | −15 | 2.2 | −4 |
| Flash point, ° C. | 129 | 168 | 127 | >110 |
| Density at | 0.79 | 0.8 | 0.78 | 0.79 |
| 20° C., g/cm³ | | | | |
| Viscosity at | 2.46 | 4.47 | 2.6 | 3.4 |
| 40° C., cSt | | | | |

*C1618 internal olefin is a mixture comprising C16 and C18 internal olefins.

The property differences clearly indicate benefit as a result of dimerization to produce vinylidene dimers.

The vinylidene dimers may be used directly as such in an application as described herein above, without further chemical modification. However, they may also be used as precursors for other chemical compounds, whereby the vinylidene dimers are chemically converted to further product or intermediates.

In one embodiment of the invention, the vinylidene dimers, i.e. one or more vinylidene dimers, are subjected to an epoxidation reaction with hydroperoxide (H₂O₂) to an epoxide, for example a C16-vinylidene-derived epoxide as represented by formula I.

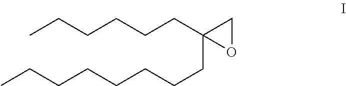 [SMILES: CCCCCCCCC1(CCCCCC)CO1]

Such vinylidene-derived epoxide is a suitable intermediate chemical for further functionalisation processes to make further products and/or further intermediates. The vinylidene-derived epoxide thus prepared has beneficial reactivity and versatility characteristics

One particular use of the intermediate vinylidene-derived epoxide is the reaction of the vinylidene-derived epoxide with sodium hydrogen sulfite to give a vinylidene-derived β-hydroxysulfonic acid, for example a C16-vinylidene-derived β-hydroxysulfonic acid as represented by formula II.

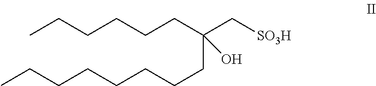 [SMILES: CCCCCCCCC(O)(CCCCCC)CS(=O)(=O)O]

Such vinylidene-derived β-hydroxysulfonic acid may find a particular application in chemical Enhanced Oil Recovery (cEOR) applications, in particular 3^{rd} generation cEOR, as surfactants.

A second particular use of the intermediate vinylidene-derived epoxide is the reaction of the vinylidene-derived epoxide with water to give a vinylidene-derived glycol, for example a C16-vinylidene-derived glycol as represented by formula III.

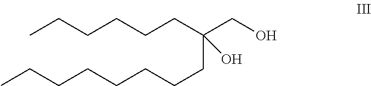 [SMILES: CCCCCCCCC(O)(CO)CCCCCC]

These vinylidene-derived glycols may be particularly suitable as an antifoaming agent. However, these vinylidene-derived glycols may also be used as an intermediate to produce ethoxylated glycols, which in turn may find a particular application again in cEOR applications, in particular 3^{rd} generation cEOR, as surfactants.

A third particular use of the intermediate vinylidene-derived epoxide is the use of the epoxide as a feedstock to produce a Guerbet alcohol by the catalytic hydrogenation of the epoxide. The obtained vinylidene-derived Guerbet alcohol may for example be a C16-vinylidene-derived Guerbet alcohol as represented by formula IV.

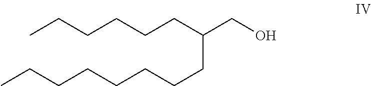 [SMILES: CCCCCCCCC(CO)CCCCCC]

Also these vinylidene-derived Guerbet alcohols may find a particular application again in cEOR applications, in particular 3^{rd} generation cEOR. However, these alcohols are also suitable for use in personal care products. These vinylidene-derived Guerbet alcohols may also be prepared by reduction via the Meerwein-Ponndorf-Verley (MPV) route using isopropanol to give a Guerbet alcohol and acetone as a byproduct. A preferred catalyst is Al(iPrO)₃.

A fourth particular use of the intermediate vinylidene-derived epoxide is the reaction of the vinylidene-derived epoxide with an acid to give a vinylidene-derived aldehyde, for example a C16-vinylidene-derived aldehyde as represented by formula V.

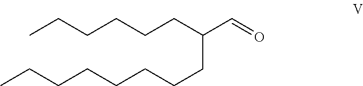 [SMILES: CCCCCCCCC(C=O)CCCCCC]

The vinylidene-derived aldehyde may be converted to acetals, preferably with monoethylglycol (MEG) for lubricant applications.

A fifth particular use of the intermediate vinylidene-derived epoxide is the reaction of the vinylidene-derived epoxide with polyethylglycol (PEG) to give a vinylidene-derived ethoxylate, for example a C16-vinylidene-derived ethoxylate as represented by formula VI.

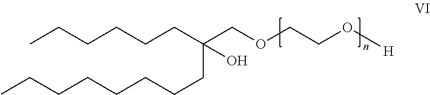

These vinylidene-derived ethoxylates may find a particular application again in cEOR applications, in particular 3^{rd} generation cEOR. The use of the vinylidene-derived epoxides has the advantage that no ethoxylation is necessary to get a hydrophilic chain (PEG) of defined length.

A sixth particular use of the intermediate vinylidene-derived epoxide is the reaction of the vinylidene-derived epoxide with amines. There are many possible reactions including but not limited to: ammonia (formula VII), diethanolamine (formula VIII), trimethylamine (formula IX) and ethylene diamine (formula X). The reaction of the vinylidene-derived epoxide with trimethylamine leads to a quaternary ammonium. Such compounds may find a particular application in cEOR, in particular 4th generation cEOR. Although, formula VIII to X show C16-vinylidene based amines, other vinylidene dimers comprising different numbers of carbon atoms may also be used.

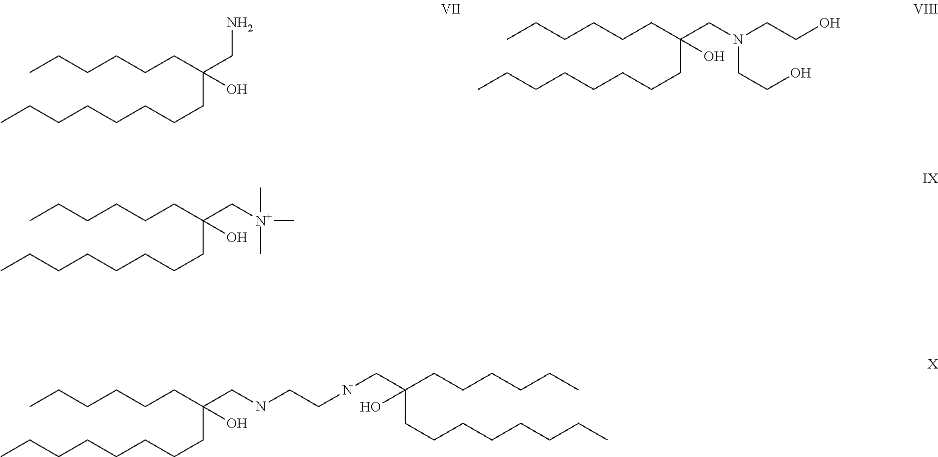 [SMILES: CCCCCCCCC(O)(CN)CCCCCC ; CCCCCCCCC(O)(CCCCCC)CN(CCO)CCO ; CCCCCCCCC(O)(CCCCCC)C[N+](C)(C)C ; CCCCCCCCC(O)(CCCCCC)CNCCNCC(O)(CCCCCC)CCCCCCCC]

In another embodiment of the invention, the vinylidene dimers are subjected to a sulfonation reaction to form β,γ-unsaturated sulfonic acid, for example a C16-vinylidene-derived β,γ-unsaturated sulfonic acid as represented by formula XI.

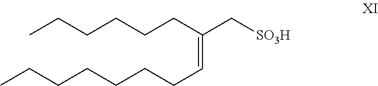 [SMILES: CCCCCCC/C=C(\CCCCCC)CS(=O)(=O)O]

Such vinylidene-derived β,γ-unsaturated sulfonic acid is a suitable intermediate chemical for further functionalisation processes to make further products and/or further intermediates. The vinylidene-derived β,γ-unsaturated sulfonic acids thus prepared may find a particular application again in cEOR applications, in particular 3^{rd} generation cEOR. For this application it is preferred that the starting vinylidene dimer is a C16, C18, or C20 vinylidene dimer.

In a third embodiment of the invention, the vinylidene dimers are subjected to a hydroformylation reaction to form alcohols, in which the hydroxymethyl group is randomly distributed along the backbone, for example a C16-vinylidene-derived alcohol as represented by formula XII.

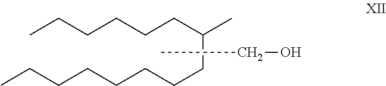

The distribution of the alcohol depends on the choice of catalyst. Such vinylidene-derived alcohols are a suitable intermediate chemical for further functionalisation processes to make further products and/or further intermediates. This vinylidene-derived alcohol may be used as an intermediate for producing surfactants by sulfonation and/or alkoxyation, preferably propoxylation.

In a fourth embodiment of the invention, the vinylidene dimers are subjected to an alkylation reaction with an aromatic compound, preferably benzene, to provide a di-substituted aromatic compound, for example a C16-vinylidene-derived di-substituted alkylbenzene as represented by formula XIII.

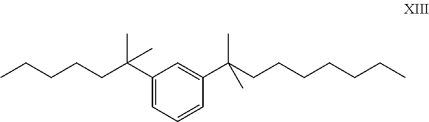 [SMILES: CCCCCCCC(C)(C)c1cccc(C(C)(C)CCCCC)c1]

Such vinylidene-derived substituted aromatic compounds may be used to provide low viscosity fluids that can be used in lubrication. Mono, di, and tri-alkylations may take place. A mixture of products with quaternary substituents (as shown) and other isomers (e.g. branched secondary alkyl substituents) may be obtained depending on the acid catalyst used. Such vinylidene-derived alkylated aromatic compounds are a suitable intermediate chemical for further functionalisation processes to make further products and/or further intermediates. Sulfonation of the aromatic ring may afford surfactants, which may be of particular use in cEOR application, in particular 4th generation cEOR. The vinylidene-derived substituted aromatic compounds are a suitable intermediate chemical for further functionalisation processes to make further products and/or further intermediates. The vinylidene-derived β,γ-unsaturated sulfonic acids thus prepared may find a particular application again in cEOR applications, in particular 3^{rd} generation cEOR. For this application it is preferred that the starting vinylidene dimer is a C16, C18, or C20 vinylidene dimer.

In a fifth embodiment of the invention, the vinylidene dimers are subjected to a carbonylation with carbon monoxide (CO), also referred to as a Koch reaction, which will give α,α-substituted with alkyl groups, for example a C16-vinylidene-derived carboxylate as represented by formula XIV.

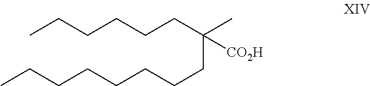 [SMILES: CCCCCCCCC(C)(CCCCCC)C(=O)O]

Such functionality gives outstanding performance when applied in outdoor coating applications. Such vinylidene-derived carboxylates are a suitable intermediate chemical for further functionalisation processes to make further products and/or further intermediates.

In a sixth embodiment of the invention, the vinylidene dimers are subjected to a reaction with maleic anhydride (MALA) to give a vinylidene-derived alkylsuccinic anhydride (ASA), which will be α,α-substituted with alkyl groups, for example a C16-vinylidene-derived alkylsuccinic anhydride as represented by formula XV.

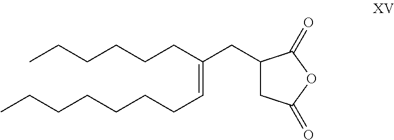 [SMILES: CCCCCCC/C=C(\CCCCCC)CC1CC(=O)OC1=O]

By using vinylidene dimers as a precursor material, a cleaner ASA product is obtained as the reaction results in less byproducts compared with the traditional olefin succinates used for paper sizing. Such vinylidene-derived alkylsuccinic anhydride may show an enhanced performance. For paper sizing, C18-vinylidene-derived alkylsuccinic anhydride is preferred; however C20-vinylidene-derived alkylsuccinic anhydride vinylidene may be used as well.

Such vinylidene-derived alkylsuccinic anhydrides are a suitable intermediate chemical for further functionalisation processes to make further products and/or further intermediates. The reactivity of the vinylidene-derived succinic anhydride enables a number of desired derivatizations.

A particular derivatization is a reaction of the vinylidene-derived succinic anhydride with amines to give succinimides, such as the one shown in formula XVI.

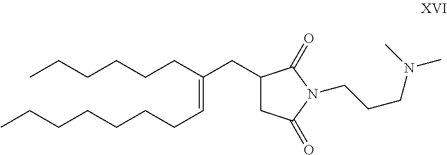 [SMILES: CCCCCCC/C=C(\CCCCCC)CC1CC(=O)N(CCCN(C)C)C1=O]

This reaction, in particular with diammonium phosphate DAP, may be used to produce lubricant additives. For instance, polyisobutylene-MALA-succinimides are well known lubricant additives; here PIB is replaced with a ‘twin-tail’ stemming from a vinylidene.

Another particular derivatization is a reaction of the vinylidene-derived succinic anhydride with amino-end-capped polyethylene glycol PEG to give compounds including the compound shown by formula XVII.

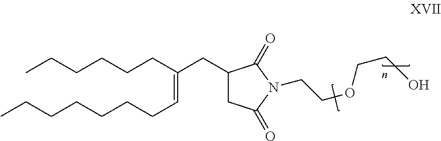

Yet another particular derivatization is a reaction of the vinylidene-derived succinic anhydride with diamines to give Zwitterionic functionality. One example of such a derivative is given by formula XVIII.

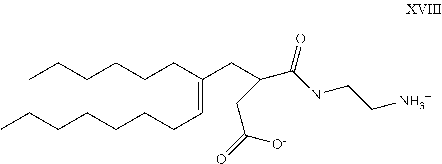 [SMILES: CCCCCCC/C=C(\CCCCCC)CC(CC(=O)[O-])C(=O)NCC[NH3+]]

These vinylidene-derived compounds may find a particular application again in cEOR applications, in particular 4^{th} generation cEOR, as they may exhibit less sensitivity to Ca²⁺. At the same time these compounds exhibit a pH tunable degradation, which may benefit demulsification.

In a seventh embodiment of the invention, the vinylidene dimers are subjected to a reaction to form vinylidene-derived silanes, for example a C16-vinylidene-derived silane as represented by formula XIX.

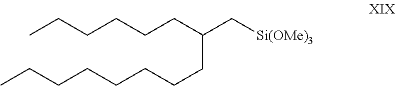

Such vinylidene-derived silanes are a suitable intermediate chemical for further functionalisation processes to make further products and/or further intermediates.

Vinylidene-derived silanes may be applied as coupling agents to improve the interface between glass fibers and polyolefin base polymers. Use of ‘twin-tail’ silanes may be attractive in view of the envisaged higher efficiency. Weight reduction is of interest in the automotive and construction industry, where steel and concrete can be replaced by composites like glass fiber re-enforced resins. For these, coupling agents are essential. This also holds for e.g. silica in reduced rolling resistance tires.

In an eighth embodiment of the invention, the vinylidene dimers are subjected to a Prins reaction with formaldehyde to form vinylidene-derived α,γ-glycol, for example a C16-vinylidene-derived α,γ-glycol as represented by formula XX.

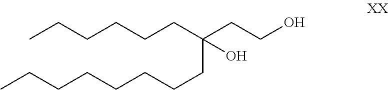 [SMILES: CCCCCCCCC(O)(CCO)CCCCCC]

Such vinylidene-derived α,γ-glycol are a suitable intermediate chemical for further functionalisation processes to make further products and/or further intermediates.

These vinylidene-derived compounds may find a particular application again in cEOR applications, in particular 4^{th} generation cEOR, as they may exhibit a pH tunable degradation, which may benefit demulsification.

In a ninth embodiment of the invention, the vinylidene dimers are subjected to a di, or trifunctionalization using one or more of the functionalizations described in the above mentioned embodiments of the invention. In one particular combination of functionalizations compounds suitable for use as anti-oxidants are formed. One example of such a compound is represented by formula XXI, although where the compound represented in formula XXI is based on a vinylidene dimer comprising 16 carbon atoms, other vinylidene dimers comprising different numbers of carbon atoms may be equally used.

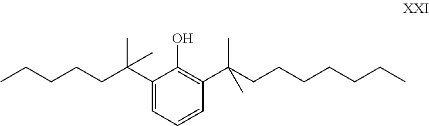 [SMILES: CCCCCCCC(C)(C)c1cccc(C(C)(C)CCCCC)c1O]

Anti-oxidants are increasingly becoming important to achieve low VOC products. The vinylidene-derived anti-oxidant of the present invention may exhibit low viscosity and low vapor pressure.

In a tenth embodiment of the invention, the vinylidene dimers are subjected to an oligomerization. These oligomerized vinylidene dimers give base lubricants with excellent performance. For example, dimerization of the C20 vinylidene would give, after hydrogenation, a C40 lubricant.

In an eleventh embodiment of the invention, the vinylidene dimers are subjected to a co-polymerisation with (a) iso-butene, (b) isobutene and 1-butene, or (c) isobutene and isoprene. The resulting compounds may be used to tune properties of polybutylene-type rubbers.

We claim:

1. A process for producing vinylidene dimer derivatives, which process includes subjecting one or more vinylidene dimers to an epoxidation reaction to obtain a vinylidene-derived epoxide.

2. The process of claim 1, wherein the one or more vinylidene dimers are epoxidated with hydrogen peroxide.

3. The process of claim 1, wherein the vinylidene-derived epoxide is subsequently reacted with sodium hydrogen sulfite to produce a vinylidene-derived β-hydroxysulfonic acid.

4. The process of claim 1, wherein the vinylidene-derived epoxide is subsequently reacted with water to produce a vinylidene-derived glycol.

5. The process of claim 1, wherein the vinylidene-derived epoxide is subsequently reacted with an acid to produce a vinylidene-derived aldehyde.

6. The process of claim 1, wherein the vinylidene-derived epoxide is subsequently catalytically hydrogenated with water to produce a vinylidene-derived Guerbet alcohol.

7. The process of claim 1, wherein the vinylidene-derived epoxide is subsequently reacted with polyethylene glycol to produce a vinylidene-derived ethoxylate.

8. The process of claim 1, wherein the vinylidene-derived epoxide is subsequently reacted with at least one of ammonia, diethanolamine, trimethylamine, and ethylene diamine.

9. A process for producing vinylidene dimer derivatives, which process includes subjecting one or more vinylidene dimers to a sulfonation reaction to obtain a vinylidene-derived β,γ-unsaturated sulfonic acid.

10. A process for producing vinylidene dimer derivatives, which process includes subjecting one or more vinylidene dimers to an alkylation reaction with an aromatic compound to obtain a vinylidene-derived di-substituted aromatic compound.

11. A process for producing vinylidene dimer derivatives, which process includes subjecting one or more vinylidene dimers to a reaction with a maleic anhydride to obtain a vinylidene-derived alkylsuccinic anhydride.

12. A process according to claim 11, wherein the maleic anhydride is a diamine and the vinylidene-derived alkylsuccinic anhydride obtained from the reaction has zwitterionic functionality.
